(19) United States
(12) Patent Application Publication
(10) Pub. No.: US 2008/0312080 A1
(43) Pub. Date: Dec. 18, 2008

(54) Novel dioctatin derivatives and production process thereof

(75) Inventors: Yasuhiko Muraoka, Shinagawa-ku (JP); Shohei Sakuda, Bunkyo-ku (JP)

Correspondence Address: Edwards Angell Palmer & Dodge LLP, P.O. Box 55874, Boston, MA, 02205, US

(73) Assignees: Microbial Chemistry Research Foundation, Tokyo (JP); The University of Tokyo, Bunkyo-ku (JP)

(21) Appl. No.: 12/222,196

(22) Filed: Aug. 5, 2008

Related U.S. Application Data
(63) Continuation of application No. PCT/JP2007/051949, filed on Feb. 5, 2007.

(30) Foreign Application Priority Data
Feb. 6, 2006 (JP) 2006-028786

(51) Int. Cl. A01N 63/02; C07C 229/26; A01P 3/00

(52) U.S. Cl. 504/117; 562/561

(57) ABSTRACT

To provide dioctatin derivatives, a production process thereof, an aflatoxin production inhibitor containing the dioctatin derivative, and a method of controlling aflatoxin contamination by use of the aflatoxin production inhibitor containing the dioctatin derivative. The present invention provides dioctatin derivatives represented by the following Structural Formula (I):

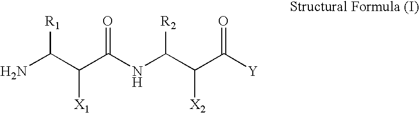

where R₁ and R₂ each represent CH₃—(CH₂)ₙ—, (CH₃)₂CH—CH₂— or C₆H₅—CH₂—; n represents an integer of 2 to 6; X₁ and X₂ each represent CH₃ or hydrogen atom; and Y represents 2-amino-2-butenoic acid or amino acid residue, with compounds where R₁ and R₂ are each CH₃(CH₂)₄—, X₂ is a hydrogen atom and Y is 2-amino-2-butenoic acid being excluded.

### CROSS-REFERENCE TO RELATED APPLICATION

This is a continuation of Application No. PCT/JP2007/051949, filed on Feb. 5, 2007.

### BACKGROUND OF THE INVENTION

1. Field of the Invention

The present invention relates to dioctatin derivatives, a production process thereof, an aflatoxin production inhibitor containing the dioctatin derivative, and a method of controlling aflatoxin contamination by use of the aflatoxin production inhibitor containing the dioctatin derivative.

2. Description of the Related Art

Dioctatin has been identified as a physiologically active substance that specifically inhibits dipeptidyl peptidase II (DPPII) and is known to be isolated from a culture of a dioctatin-producing microorganism (Streptomyces avermitilis, sp. SA-2581) (see Japanese Patent (JP-B) No. 2966859).

JP-B No. 2966859 discloses the physical properties and planar structure of dioctatin, but fails to reveal its three-dimensional structure. The dioctatin compounds disclosed by this document have the following Structural Formula (1):

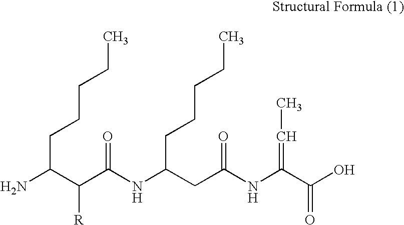

where R denotes a hydrogen atom or methyl group.

Dioctatin compounds covered by the above Structural Formula (1) are dioctatin A, a dioctatin with R being methyl group, containing three asymmetric carbons, and dioctatin B, a dioctatin with R being hydrogen atom, containing two asymmetric carbons. JP-B No. 2966859, however, fails to disclose their absolute structures.

Incidentally, secondary metabolites of fungi are known to contain useful compounds, but contain many toxic compounds called mycotoxins as well. Mycotoxin contamination of farm crops has been a serious global problem, and therefore, measures to control mycotoxin contamination have been demanded for stable provision of safe foods.

Aflatoxin contamination of farm crops is the most severe of all types of mycotoxin contamination. Aflatoxin is known as a most potent cancer-causing agent of all known naturally occurring substances and is a compound not decomposable by a normal cooking method. For these reasons, the upper limit for aflatoxin contamination level is set to as low as 10 ppb. Moreover, damage costs incurred by disposal of aflatoxin-contaminated farm crops has been increasing.

Since aflatoxin is a second metabolite, it is considered that inhibition of its production dose not influence the growth of the aflatoxin-producing microorganism. Thus, the present inventors contemplated that utilization of a compound that specifically inhibits aflatoxin production may be a potent method of contamination control, and screening of known physiologically active substances led to the identification of dioctatin as a compound that inhibits production of aflatoxin without inhibiting the growth of aflatoxin-producing microorganism (see Japanese Patent Application Laid-Open (JP-A) No. 2007-31419).

As described above, this document reveals that dioctatin is useful as a compound with a DPPII-inhibiting activity and as a compound with an aflatoxin production-inhibiting activity.

Unfortunately, the production process of dioctatin by isolation from a culture of dioctatin-producing microorganism has such disadvantages as unstable dioctatin yield and complicate purification process. Production of dioctatin by chemical synthesis, on the other hand, was difficult since the absolute structure of dioctatin had not been elucidated.

To overcome this problem, in JP-A No. 2007-31419, the present inventors revealed the three-dimensional structure of naturally occurring dioctatin by synthesizing several stereoisomers based on the planar structure of naturally occurring dioctatin obtained from a culture of dioctatin-producing microorganism and by comparing their physical properties.

It is expected that novel compounds, which would show DPPII-inhibiting activity and aflatoxin production-inhibiting activity that are comparable or greater than those of naturally occurring dioctatins, are present in the stereoisomers and derivatives of dioctatin obtained in the course of dioctatin synthesis. However, the current situation is that such novel dioctatin derivatives and production process thereof have not yet been provided.

### BRIEF SUMMARY OF THE INVENTION

An object of the present invention is to solve the foregoing problems pertinent in the art and to achieve the following object.

More specifically, it is an object of the present invention to provide novel dioctatin derivatives, a production process thereof, and among the novel dioctatin derivatives, novel dioctatin derivatives useful as DPPII inhibitors that specifically inhibit DPPII, novel dioctatin derivatives useful as aflatoxin production inhibitors that specifically and effectively inhibit production of aflatoxin, and a method of controlling aflatoxin contamination by use of the aflatoxin production inhibitor containing the dioctatin derivative.

The present inventors extensively conducted studies to overcome the above problems and reached the following finding: In the course of establishing a production process of naturally occurring dioctatins (dioctatin A and dioctatin B) by chemical synthesis, stereoisomers and novel similar-structure derivatives of dioctatin were produced, and evaluation of their aflatoxin production-inhibiting activity and DPPII-inhibiting activity established that compounds that offer physiological activities comparable or greater than those of the naturally occurring dioctatins are present among them, and that such novel dioctatin derivatives can be produced readily and effectively, as can naturally occurring dioctatins.

The present invention has been accomplished based on the foregoing findings by the present inventors, and means of solving the foregoing problems are as follows:

<1> Dioctatin derivatives represented by the following Structural Formula (I):

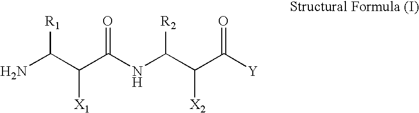

where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; and Y represents 2-amino-2-butenoic acid or amino acid residue, with compounds where R₁ and R₂ are each CH₃(CH₂)₄—, X₂ is a hydrogen atom and Y is 2-amino-2-butenoic acid being excluded.

<2> The dioctatin derivatives according to <1>, wherein the dioctatin derivatives are represented by the following Structural Formula (II):

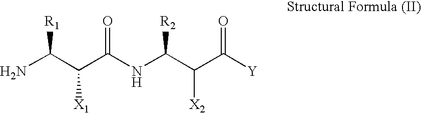

where R₁ and R₂ each represent CH₃—(CH₂)ₙ—, (CH₃)₂CH—CH₂— or C₆H₅—CH₂—; n represents an integer of 2 to 6; X₁ represents a hydrogen atom or CH₃; X₂ represents a hydrogen atom; and Y represents 2-amino-2-butenoic acid or amino acid residue, with compounds where R₁ and R₂ are each CH₃(CH₂)₄— and Y is 2-amino-2-butenoic acid being excluded.

<3> The dioctatin derivatives according to one of <1> and <2>, wherein the amino acid residue is selected from the group consisting of glycine residue, sarcosine residue, L-alanine residue, β-alanine residue, L-proline residue, L-valine residue, L-leucine residue, L-phenylalanine residue, L-thioproline residue, and 4-hydroxy-L-proline residue.

<4> A production process of dioctatin derivative including:

condensing a compound represented by the following Structural Formula (b) with an amino acid derivative in which its carboxyl group is protected, to prepare a dipeptide compound;

removing a protective group of an amino group of the dipeptide compound;

condensing the dipeptide compound with a compound represented by the following Structural Formula (a) to prepare a tripeptide compound; and

removing a protective group of the tripeptide compound.

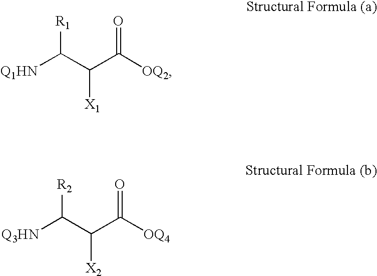

where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ and Q₃ each represent Boc group, carbobenzoxy group, p-methoxybenzyloxycarbonyl group, Fmoc group, 2,2,2-trichloroethoxycarbonyl group, or allyloxycarbonyl group; and Q₂ and Q₄ each represent a hydrogen atom.

<5> A production process of dioctatin derivative including:

condensing a compound represented by the following Structural Formula (a) with a compound represented by the following Structural Formula (b), to prepare a dipeptide compound;

removing a protective group of a carboxyl group of the dipeptide compound;

condensing the dipeptide compound with an amino acid derivative in which its carboxyl group is protected, to prepare a tripeptide compound; and

removing a protective group of the tripeptide compound.

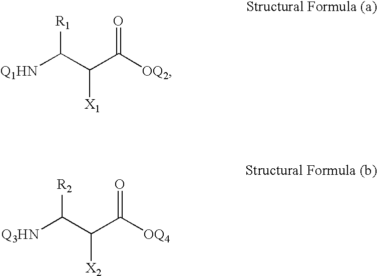

where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ represents Boc group, carbobenzoxy group, p-methoxybenzyloxycarbonyl group, Fmoc group, 2,2,2-trichloroethoxycarbonyl group, or allyloxycarbonyl group; Q₂ and Q₃ each represent a hydrogen atom; and Q₄ represents a hydrogen atom, methyl group, ethyl group, benzyl group, t-butyl group, or 2,2,2-trichloroethyl group.

<6> The production process according to one of <4> and <5>, wherein the compounds represented by Structural Formulas (a) and (b) have S configuration at 3-position.

<7> The production process according to any one of <4> to <6>, wherein in Structural Formula (a) X₁ represents CH₃, and the compounds represented by Structural Formula (a) have R configuration.

<8> The production process according to any one of <4> to <7>, wherein the amino acid derivative is selected from the group consisting of glycine, sarcosine, L-alanine, β-alanine, L-proline, L-valine, L-leucine, L-phenylalanine, L-thioproline, and 4-hydroxy-L-proline.

<9> An aflatoxin production inhibitor including a dioctatin derivative according to any one of <1> to <3>.

<10> The aflatoxin production inhibitor according to <9>, wherein the dioctatin derivative is represented by the following Structural Formula (III):

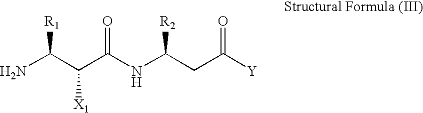

where R₁ and R₂ each represent CH₃—(CH₂)ₙ— or (CH₃)₂CH—CH₂—; X₁ represents a hydrogen atom or CH₃; n represents an integer of 2 to 6; and Y represents an amino acid residue.

<11> A method of aflatoxin contamination control including:

inhibiting aflatoxin production by aflatoxin-producing microorganism by use of an aflatoxin production inhibitor according to one of <9> and <10>.

<12> The method of aflatoxin contamination control according to <11>, wherein the aflatoxin production inhibitor is applied to a farm crop for inhibition of production of aflatoxin from aflatoxin-producing microorganism that infected the crop.

According to the present invention, it is possible to solve the problems pertinent in the art and to provide novel dioctatin derivatives, a production process thereof, novel dioctatin derivatives useful as aflatoxin production inhibitors, and a method of controlling aflatoxin contamination by use of the aflatoxin production inhibitor containing the dioctatin derivative.

### DETAILED DESCRIPTION OF THE INVENTION

### (Dioctatin Derivatives)

Dioctatin derivatives of the present invention are compounds represented by the following Structural Formula (I):

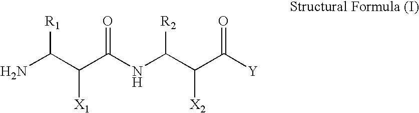

where R₁ and R₂ each represent CH₃—(CH₂)ₙ—, (CH₃)₂CH—CH₂—, or C₆H₅—CH₂—; n represents an integer of 2 to 6; X₁ and X₂ each represent CH₃ or hydrogen atom; and Y represents 2-amino-2-butenoic acid or amino acid residue.

Note in the Structural Formula (I) above that a compound wherein R₁ and R₂ are each CH₃(CH₂)₄—, X₂ is hydrogen atom and Y is 2-amino-2-butenoic acid, i.e., the compound represent represented by the following Structural Formula (2) is excluded.

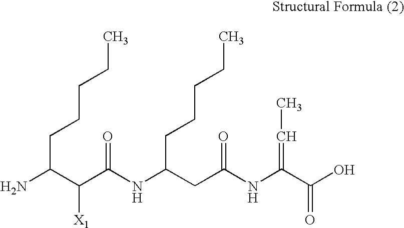

Among compounds covered by the above Structural Formula (I), compounds having three-dimensional structure represented by the following Structural Formula (II) are preferable.

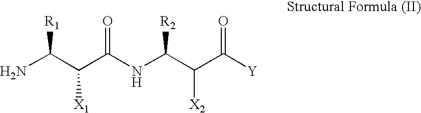

where R₁ and R₂ each represent CH₃—(CH₂)ₙ—, (CH₃)₂CH—CH₂— or C₆H₅—CH₂—; n represents an integer of 2 to 6; X₁ represents a hydrogen atom or CH₃; X₂ represents a hydrogen atom; and Y represents 2-amino-2-butenoic acid or amino acid residue.

Note in the Structural Formula (II) above that a compound wherein R₁ and R₂ are each CH₃(CH₂)₄— and Y is 2-amino-2-butenoic acid, i.e., the compound represent represented by the following Structural Formula (3) is excluded.

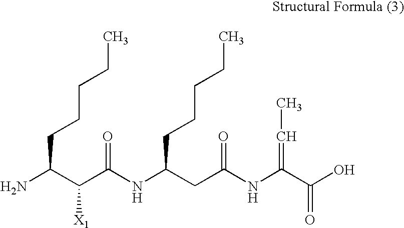

The amine acid residue is not particularly limited and can be appropriately selected depending on the intended purpose; preferable examples include, for example, residues of glycine, sarcosine, L-alanine, β-alanine, L-proline, L-valine, L-leucine, L-phenylalanine, L-thioproline, and 4-hydroxy-L-proline, with residues of glycine, L-alanine, and L-proline being more preferable.

The dioctatin derivatives represented by the above Structural Formulas (I) and (II) are produced by a later-described production process of the present invention for producing dioctatin derivatives.

The dioctatin derivatives represented by the above Structural Formulas (I) and (II) are preferably physiologically active substances that show aflatoxin production-inhibiting activity.

### <Aflatoxin Production Inhibitor>

An aflatoxin production inhibitor of the present invention is not particularly limited as long as it contains as an active ingredient a dioctatin derivative represented by Structural Formula (II), and may contain additional ingredient(s) appropriately selected, e.g., carriers, depending on the intended purpose.

The dosage form of the aflatoxin production inhibitor is not particularly limited and can be appropriately determined depending on the intended purpose; examples are formulations prepared using known carries for use for pharmaceutical agents and agricultural and gardening formulations; examples of formulations include, for example, solid formulations, powders, tablets, capsules, granules, liquids, gels, creams, and sprays.

The aflatoxin production inhibitor of the present invention is suitable for use in a method of aflatoxin contamination control to be described later.

Examples of dioctatin derivatives suitable as an ingredient in the aflatoxin production inhibitor include, for example, (S)-3-aminooctanoyl-(S)-3-aminooctanoyl-L-proline, (S)-3-aminooctanoyl-(S)-3-aminodecanoyl-L-proline, (S)-3-aminohexanoyl-(S)-3-aminooctanoyl-L-proline, (S)-3-aminooctanoyl-(S)-3-aminooctanoyl-glycine, (S)-3-aminooctanoyl-(S)-3-aminodecanoyl-glycine, (S)-3-aminohexanoyl-(S)-3-aminooctanoyl-glycine, (S)-3-amino-5-methylhexanoyl-(S)-3-aminooctanoyl-glycine, (S)-3-aminooctanoyl-(S)-3-aminooctanoyl-L-alanine, (S)-3-aminooctanoyl-(S)-3-aminooctanoyl-L-valine, (S)-3-aminooctanoyl-(S)-3-aminooctanoyl-L-leucine, (S)-3-aminooctanoyl-(S)-3-aminooctanoyl-L-phenylalanine, (S)-3-aminooctanoyl-(S)-3-aminooctanoyl-β-alanine, (S)-3-aminooctanoyl-(S)-3-aminooctanoyl-sarcosine, (2R,3S)-3-amino-2-methyloctanoyl-(S)-3-aminooctanoyl-L-proline, and (2R,3S)-3-amino-2-methyloctanoyl-(S)-3-aminooctanoyl-glycine.

Among them, dioctatin derivatives represented by the following Structural Formula (III) are preferable.

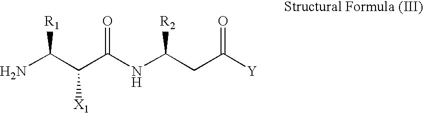

where R₁ and R₂ each represent CH₃—(CH₂)ₙ— or (CH₃)₂CH—CH₂—; X₁ represents a hydrogen atom or CH₃; n represents an integer of 2 to 6; and Y represents an amino acid residue.

Table 1 below lists examples of preferable dioctatin derivatives. Note in Table 1 that R₁, R₂, X₁, X₂ and Y respectively correspond to R₁, R₂, X₁, X₂ and Y in Structural Formula (II).

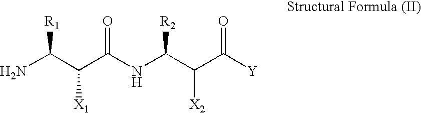

TABLE 1

| | R₁ | X₁ | R₂ | X₂ | Y |
|---|---|---|---|---|---|
| 1 | CH₃(CH₂)ₙ— | CH₃[R] | CH₃(CH₂)ₙ— | H | glycine |
| | (S, n = 4) | | (S, n = 4) | | |
| 2 | CH₃(CH₂)ₙ— | H | CH₃(CH₂)ₙ— | H | glycine |
| | (S, n = 2) | | (S, n = 4) | | |
| 3 | (CH₃)₂CHCH₂— | H | CH₃(CH₂)ₙ— | H | glycine |
| | | | (S, n = 4) | | |
| 4 | CH₃(CH₂)ₙ— | H | CH₃(CH₂)ₙ— | H | glycine |
| | (S, n = 4) | | (S, n = 4) | | |
| 5 | CH₃(CH₂)ₙ— | H | CH₃(CH₂)ₙ— | H | sarcosine |
| | (S, n = 4) | | (S, n = 4) | | |
| 6 | CH₃(CH₂)ₙ— | H | CH₃(CH₂)ₙ— | H | L-alanine |
| | (S, n = 4) | | (S, n = 4) | | |
| 7 | CH₃(CH₂)ₙ— | H | CH₃(CH₂)ₙ— | H | β-alanine |
| | (S, n = 4) | | (S, n = 4) | | |
| 8 | CH₃(CH₂)ₙ— | H | CH₃(CH₂)ₙ— | H | L-proline |
| | (S, n = 4) | | (S, n = 4) | | |
| 9 | CH₃(CH₂)ₙ— | H | CH₃(CH₂)ₙ— | H | glycine |
| | (S, n = 4) | | (S, n = 6) | | |

In Table 1 “S” represents S configuration and “R” represents R configuration.

### (Method of Aflatoxin Contamination Control)

A method aflatoxin contamination control of the present invention is a method of inhibiting production of aflatoxin by aflatoxin-producing microorganism by use of the aflatoxin production inhibitor of the present invention, and is not particularly limited as long as it is a method of applying the aflatoxin production inhibitor to a target to which aflatoxin-producing microorganism is attached or which is infected with the aflatoxin-producing microorganism, and can be appropriately selected depending on the intended purpose.

Examples of the target include, for example, vegetables and farm crops. Examples of farm crops include, for example, grains such as corn, rice, buckwheat and wheat; nuts such as peanuts, pistachio peanuts and Brazil peanuts; spices such as nutmeg seed, hot pepper and paprika; and beans such as coffee beans.

The method of applying the aflatoxin production inhibitor to a target to which aflatoxin-producing microorganism is attached or which is infected with the aflatoxin-producing microorganism is not particularly limited and can be appropriately selected depending on the intended purpose. For example, the aflatoxin production inhibitor is formulated into normal dosage form (e.g., agricultural formulation) and applied to such a target by coating or spraying.

The concentration of the dioctatin derivative in the aflatoxin production inhibitor used in the method of aflatoxin contamination control is adjusted according to the type and/or propagation of the aflatoxin-producing microorganism; for example, it is preferably 10 ppm to 50,000 ppm, more preferably 100 ppm to 5,000 ppm.

### (Production Process of Dioctatin Derivatives)

A production process of the present invention for producing dioctatin derivatives is of two forms: (1) A first form in which the dioctatin derivative represented by Structural Formula (II) is synthesized from the C-terminal side, and (2) a second form in which the dioctatin derivative represented by Structural Formula (II) is synthesized from the N-terminal side.

### <First Form>

The first form of the production process includes the steps of condensing a compound represented by the following Structural Formula (b) with an amino acid derivative in which its carboxyl group is protected, to prepare a dipeptide compound; removing the protective group of the amino group of the dipeptide compound; condensing it with a compound represented by the following Structural Formula (a) to prepare a tripeptide compound; and removing the protective group of the tripeptide compound.

The condensation method and removal method of protective group are not particularly limited and can be appropriately selected from those known in the art.

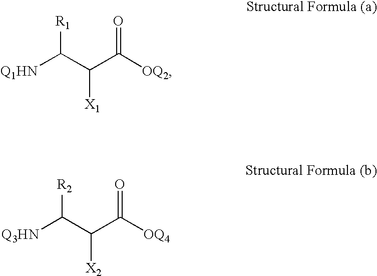

where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ and Q₃ each represent Boc group, carbobenzoxy group, p-methoxybenzyloxycarbonyl group, Fmoc group, 2,2,2-trichloroethoxycarbonyl group, or allyloxycarbonyl group; and Q₂ and Q₄ each represent a hydrogen atom.

Chemical synthesis of naturally occurring dioctatins cannot employ contact reduction in the final protective group removal step, since the compound contains a unsaturated amino acid. By contrast, chemical synthesis of amino acid-substituted dioctatin derivatives among dioctatin derivatives of the present invention can employ contact reduction.

More specifically, an amino acid derivative in which its carboxyl group is protected (e.g., amino acid benzyl ester) is condensed with a compound represented by Structural Formula (b) (e.g., 3-aminoalkanoic acid in which the amine is protected, such as a Boc-protected 3-aminoalkanoic acid); when the resultant compound is protected by the Boc group, it is removed by treatment with TFA or hydrochloric acid/dioxane, and a compound represented by Structural Formula (a) (e.g., 3-aminoalkanoic acid in which the amine is protected, such as Boc-protected 3-aminoalkanoic acid) is further condensed with the compound to form a protected tripeptide benzyl ester; and when the compound is protected by the Boc group, it is removed by treatment with TFA, and benzyl ester is removed by contact reduction as a final deprotection process to obtain a novel dioctatin derivative of the present invention represented by Structural Formula (1).

The amino acid derivative in which its carboxyl group is protected is not particularly limited and can be appropriately selected depending on the intended purpose; examples include, for example, amino acid benzyl ester p-toluenesulfonates, with glycine benzyl ester p-toluenesulfonate, L-alanine benzyl ester p-toluenesulfonate, L-valine benzyl ester p-toluenesulfonate and the like being preferable.

The compound represented by Structural Formula (a) is not particularly limited and can be appropriately selected depending on the intended purpose; preferable examples include, for example, Boc-(S)-3-aminooctanoic acid, Boc-(S)-3-aminohexanoic acid, Boc-(S)-3-amino-5-methylhexanoic acid, and Boc-(2R,3S)-3-amino-2-methyloctanoic acid.

The compound represented by Structural Formula (b) is not particularly limited and can be appropriately selected depending on the intended purpose; preferable examples include, for example, Boc-(S)-3-aminooctanoic acid, and Boc-(S)-3-aminodecanoic acid.

The dioctatin derivative has a B-amino acid as the center amino acid of the tripeptide, and therefore, there is no risk that it undergoes racemization that may occur upon peptide synthesis using normal α-amino acids. For this reason, the dioctatin derivative can be produced not only through the first form where the peptide chain grows from C-terminal to N-terminal, but through the second form to be described later, where a third amino acid is condensed with the N-terminal dipeptide.

### <Second Form>

The second form of the production process of the dioctatin derivative includes the steps of condensing a compound represented by the following Structural Formula (a) with a compound represented by the following Structural Formula (b), to prepare a dipeptide compound; condensing the dipeptide compound with an amino acid derivative in which its carboxyl group is protected, to prepare a tripeptide compound; and removing the protective group of the tripeptide compound.

The condensation method and removal method of protective group are not particularly limited and can be appropriately selected from those known in the art.

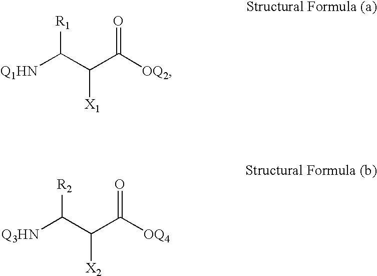

where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ represents Boc group, carbobenzoxy group, p-methoxybenzyloxycarbonyl group, Fmoc group, 2,2,2-trichloroethoxycarbonyl group, or allyloxycarbonyl group; Q₂ and Q₃ each represent a hydrogen atom; and Q₄ represents a hydrogen atom, methyl group, ethyl group, benzyl group, t-butyl group, or 2,2,2-trichloroethyl group.

A compound represented by Structural Formula (a) (e.g., ethyl 3-aminoalkanoate such as ethyl 3-aminodecanoate, ethyl 3-aminononoate, ethyl 3-aminoheptanoate or ethyl 3-aminohexanoate, which are intermediates generated upon synthesis of 3-aminoalkanoic acid) is condensed with a compound represented by Structural Formula (b) (e.g., Boc-protected 3-aminoalkanoic acid), the resultant Boc-dipeptide ethyl ester is saponified, the saponified Boc-dipeptide ethyl ester is condensed with an amino acid derivative in which its carboxyl group is protected (e.g., t-butyl ester of glycine, sarcosine, L-alanine, L-proline, or β-alanine) to form a Boc-tripeptide-t-butyl ester, and the Boc group and t-butyl ester group are removed at a time by treatment with TFA or hydrochloric acid/dioxane solution to produce a novel dioctatin derivative of the present invention represented by Structural Formula (1).

The amino acid derivative in which its carboxyl group is protected is not particularly limited and can be appropriately selected depending on the intended purpose; preferable examples include, for example, amino acid t-butyl ester hydrochlorides, with L-proline t-butyl ester, glycine t-butyl ester hydrochloride, L-alanine t-butyl ester hydrochloride, β-alanine t-butyl ester hydrochloride, sarcosine t-butyl ester hydrochloride, L-valine t-butyl ester hydrochloride, L-leucine t-butyl ester hydrochloride, L-phenylalanine t-butyl ester hydrochloride and the like being more preferable.

The compound represented by Structural Formula (a) is not particularly limited and can be appropriately selected depending on the intended purpose; preferable examples include, for example, ethyl (S)-3-aminooctanoate, and ethyl (S)-3-aminodecanoate.

The compound represented by Structural Formula (b) is not particularly limited and can be appropriately selected depending on the intended purpose; preferable examples include, for example, Boc-(S)-3-aminooctanoic acid, Boc-(2R,3S)-3-amino-2-methyloctanoic acid, and Boc-(S)-3-aminohexanoic acid.

### EXAMPLES

Hereinafter, the present invention will be described with reference to Examples, which however shall not be construed as limiting the scope of the present invention.

### Example 1

### Production of (2R,3-3-amino-2-methyloctanoyl-(S)-4-aminooctanoyl-glycine

Boc-(2R,3S)-3-amino-2-methyloctanoic acid, Boc-3-aminooctanoic acid, and Boc-(S)-3-aminooctanoyl glycine benzyl ester were synthesized. Using these compounds, (2R,3S)-3-amino-2-methyloctanoyl-(S)-3-aminooctanoyl-glycine was produced in the manner described below.

### [1] Preparation of Boc-(2R,3S)-3-amino-2-methyloctanoic acid

### [1-1] Preparation of ethyl 2-methyl-2-octenoate

Under nitrogen atmosphere, 3201.5 mg (67.51 mmol) of sodium hydride (oil content=50%) was washed with n-hexane, 80.0 mL of anhydrous tetrahydrofuran (THF) was added, and cooled to 0° C. in an ice bath. To the resultant solution was gradually added 15.0 mL (69.85 mmol) of ethyl diethylphosphonoacetate and stirred for 1 hour. Subsequently, 6148.3 mg (61.38 mmol) of n-hexanol in 10.0 mL of anhydrous THF was added and stirred for 1 hour while raising its temperature to room temperature. The reaction was quenched by addition of 200 mL of water, followed by extraction with ethyl acetate (50 mL×5 times). The organic phase was dried with magnesium sulfate, the solvent was distilled off, and the resultant oily residue was purified by column chromatography (silica gel 60N=60 g, ethyl acetate/n-hexane (1:9) ethyl acetate/n-hexane (1:4)) to produce 10557.0 mg of ethyl 2-methyl-2-octenoate (E-Z=6:1) at a yield of 93%.

[1-2] Preparation of ethyl N-benzyl-N-(1-phenylethylamino)-3-amino-2-methyloctanoate

Under nitrogen atmosphere, 24.0 mL of 1.58 M n-butyl lithium hexane solution (37.92 mmol) was gradually added to 8.1 mL (41.45 mmol) of (S)-(−)-N-benzyl-1-phenylethylamine in 40.5 mL of toluene cooled to 0° C. in an ice bath, and stirred for 20 minutes. The reaction solution was cooled to −78° C. in an ice-acetone bath, 20.0 mL of toluene solution of 4715.8 mg (25.9 mmol) of ethyl 2-methyl-2-octenoate obtained [1-1] was added, and stirred for 3 hours.

Subsequently, 180.0 mL of anhydrous tetrahydrofuran (THF) and 20 mL of anhydrous THF solution of 15011.5 mg (72.75 mmol) of 2,6-di-tert-butylphenol were sequentially added to the resultant solution, and stirred for 1 hour while raising its temperature to room temperature. The solvent of the reaction solution was then distilled off and 200 mL of water was added, followed by extraction with ethyl acetate (100 mL×4 times). The organic phase was dried with magnesium sulfate, the solvent was distilled off, and the resultant oily residue was purified by column chromatography (silica gel 60N=100 g, n-hexane→ether/n-hexane (6:94)) to produce 2535.2 mg of ethyl N-benzyl-N-(1-phenylethylamino)-3-amino-2-methyloctanoate at a yield of 25%.

### [1-3] Preparation of (2R,3S)-3-amino-2-methyloctanoic acid

To 2106.5 mg (5.33 mmol) of ethyl N-benzyl-N-(1-phenylethylamino)-3-amino-2-methyloctanoate prepared [1-2] in 30.0 mL of methanol was added 2.6 mL of water and 1.6 mL of acetic acid. Thereafter, 261.5 mg of palladium (II) hydroxide/activated charcoal (20%) was added and, after purging the reaction vessel with hydrogen, stirred for 16 hours at room temperature. The resultant solution was filtrated and the solvent was distilled off, producing a crude product of an ethyl ester of amino acid. 30.0 mL of 4 M hydrochloric acid was then added to the product, heated to 85° C., and stirred for 20 hours. The reaction solution was cooled to room temperature, 210.0 mL of water was added, and chlorine ions were removed by ion-exchange resin (Dowex, 50 w×2, Φ22 mm×260 mm) to produce a crude crystal of (2R,3S)-3-amino-2-methyloctanoic acid.

The above procedure was repeated using 2115.4 mg (5.34 mmol) of N-benzyl-N-(1-phenylethylamino)-3-amino-2-methyloctanoate obtained in [1-2], to similarly produce a crude crystal of (2R,3S)-3-amino-2-methyloctanoic acid. Subsequently, 2334.6 mg of the crude crystal of (2R,3S)-3-amino-2-methyloctanoic acid obtained above was dissolved in a mixture solvent of methanol and ethyl acetate, and purified by recrystallization to produce 1.367.8 mg of highly pure (2R,3S)-3-amino-2-methyloctanoic acid (primary crystal=856.8 mg, secondary crystal=230.5 mg, third crystal=280.5 mg) at a yield of 74%. The analytical values are shown below.

[α]²⁵_{D}+5.28 (c 1.05, MeOH)

ESI MS m/z 174.15[M+H]⁺

¹H NMR (D₂O, 600 MHz)

δ 0.90 (3H, t-like, J=7.8 Hz, H-8), 1.19 (3H, d, J=7.6 Hz, H-9), 1.34 (4H, m, H-6, 7), 1.39 (1H, m, H-5a), 1.44 (1H, m, H-5b), 1.66 (2H, m, H-4), 2.60 (1H, dq, J=5.2, 7.6 Hz, H-2), 3.43 (1H, dt, J=7.4, 5.2 Hz, H-3)

¹³C NMR (D₂O, 600 MHz) δ 14.7, 15.8, 21.0, 35.0, 45.6, 56.1, 185.1

### [1-4] Preparation of Boc-(2R,3S)-3-amino-2-methyloctanoic acid

420 mg of (2R,3S)-3-amino-2-methyloctanoic acid obtained in [1-3] was dissolved in 2.1 mL of water and 2.1 mL of dioxane, and 2.1 mL of 1 M NaOH and 504 mg of Boc₂O in 2.1 mL of dioxane were alternately added under ice-cold conditions with stirring. After stirring for 1 hour at room temperature, the solution was concentrated under reduced pressure, the pH value was adjusted to 3 by addition of 5% KHSO₄ aqueous solution, and extracted with ethyl acetate 3 times. The solution obtained after the extraction was washed with water, dried with anhydrous sodium sulfate, and concentrated under reduced pressure. The resultant residue was kept cold overnight to dryness. In this way 238 mg of Boc-(2R,3S)-3-amino-2-methyloctanoic acid was produced. The analytical values are shown below.

[α]²⁵_{D}=−14.58 (c=1, EtOAc)

¹H NMR (CDCl₃, 600 MHz)

δ 0.88 (3H, t, J=6.9), 1.17 (3H, d, J=7.2 Hz), 1.31 (6H, m), 1.44 (9H, s) Boc, 1.51 (2H, m) 4-CH₂, 2.68 (1H, br.s) 2-CH₂, 3.80 (1H, br.s) 3-CH—NHBoc, 4.74 (1H, br.s) NH

### [2] Preparation of Boc-(S)-3-aminooctanoyl-glycine benzyl ester

### [2-1] Preparation of (S)-3-aminooctanoic acid

10 mL of (S)-N-benzyl-1-phenylethylamine was dissolved in 150 mL of dehydrated tetrahydrofuran, cooled with dry ice-acetone, and 28 mL of 1.6 M butyl lithium hexane solution was added dropwise thereto in a nitrogen stream. After stirring for 30 minutes under cold conditions by dry ice-acetone, 5.2 mL of ethyl 2-octenoate was dissolved in 20 mL of tetrahydrofuran, and the resultant solution was added dropwise and stirred for 2 hours under cold conditions by dry ice-acetone. Subsequently, 40 mL of saturated aqueous solution of antimony chloride was added and stirred. The reaction solution was concentrated with a rotary evaporator for removal of large portion of tetrahydrofuran, followed by extraction with chloroform twice. The chloroform solution was dried with anhydrous sodium sulfate and concentrated to produce a mixture of adduct and excess (S)-N-benzyl-1-phenylethylamine. The mixture was then dissolved in hexane and injected in a 200 mL-silica gel column packed with hexane, eluted first with hexane and then with hexane/ether (50:3), and the first fraction that showed UB absorption was collected and concentrated to yield 6.2 g of adduct.

The resultant adduct was dissolved in a mixture of 16 mL of water, 4 mL of acetic acid and 80 mL of methanol, 880 mg of 10% palladium hydroxide-carbon was added, and reduction was effected for 16 hours at a hydrogen pressure of 40 psi. In this way ethyl 3-aminooctanoate was produced. The catalyst was filtered off, the residue was concentrated, and 60 mL of 4 N hydrochloric acid was added for hydrolysis for 16 hours at 80° C. The reaction solution was concentrated for removal of large portion of hydrochloric acid, dissolved in water and adsorbed to a 100 mL column of ion-exchange resin Dowex 50 (H type). After washing with water, the column was eluted with 2 N ammonium water and the eluate was fractioned. The ninhydrin-positive fraction was collected and concentrated to dryness, producing 1.9 g of a clear solid of (S)-3-aminooctanoic acid. The analytical values are shown below.

[α]²¹_{D}+29.1 (c=1, H₂O)

Literature's value: [α]²¹_{D}+31.1 (c=1.11, H₂O) Angew. Chem. Int. Ed. vol. 34, pp. 455-456 (1995).

NMR (D₂O, 400 MHz)

δ 0.7 (3H, t), 1.1-1.3 (6H, m), 1.5 (2H, q), 2.25 (1H, q), 2.4 (1H, q), 3.3 (1H, m)

### [2-2] Preparation of (S)-N-Boc-3-aminooctanoic acid

930 mg of (S)-3-aminooctanoic acid obtained in [2-1] was dissolved in 5.84 mL of water and 5.84 mL of dioxane, and 5.84 mL of 1 M NaOH and 1407 mg of Boc₂O in 5.84 mL of dioxane were alternately added under ice-cold conditions with stirring. After stirring for 1 hour at room temperature, the solution was concentrated under reduced pressure, the pH value was adjusted to 3 by addition of 5% KHSO₄ aqueous solution, and extracted with ethyl acetate 3 times. The solution obtained after the extraction was washed with water, dried with anhydrous sodium sulfate, and concentrated under reduced pressure. The resultant residue was kept cold overnight to dryness. In this way 1472 mg of (S)-N-Boc-3-aminooctanoic acid was produced. The analytical values are shown below.

¹H NMR (CDCl₃, 600 MHz)

δ 0.87 (3H, t-like, J=6.9 Hz, H-8), 1.22-1.37 (6H, m), 1.43 (9H, Boc), 1.51 (2H, q, H-4), 2.55 (2H, m, H-2), 3.89 (1H, m H-3)

### [2-3] Preparation of Boc-(S)-3-aminooctanoyl-glycine benzyl ester

300 mg of Boc-(S)-3-aminooctanoic acid obtained in [2-2], 430 mg of p-toluenesulfonate of glycine benzyl ester, 562 mg of Bop reagent, and 172 mg of HOBT were dissolved in 2 mL of DMF, 0.518 mL of TEA was added, and stirred overnight. 50 mL of ethyl acetate was added to the reaction solution, the solution was washed with 10% citric acid aqueous solution, 4% sodium acid carbonate aqueous solution, and saturated salt water, dried with anhydrous sodium sulfate, and concentrated to dryness to yield 466 mg of Boc-(S)-3-aminooctanoyl-glycine benzyl ester. The analytical values are shown below.

¹H NMR (DMSO-6d, 600 MHz)

δ 0.87 (3H, t, J=7.0), 1.27 (6H, m), 1.43 (9H, s) Boc, 1.51 (2H, br-q, J=7.2) 4-CH₂, 2.43 (1H, dd, J=5.8, 14.9), 2.46 (1H, Br d, J=13.9), 3.84 (1H, m), 4.06 (2H, AB type), 5.08 (1H, br.s) NH, 5.18 (2H, s) —O—CH₂-Ph, 6.35 (1H, br.s) NH, 7.36 (5H, m) Ph

### [3] Production of (2R,3S)-3-amino-2-methyloctanoyl-(S)-3-aminooctanoyl-glycine

221 mg of Boc-(S)-3-aminooctanoyl-glycine benzyl ester obtained in [2] was dissolved in 4 mL of trifluoroacetic acid, and the solution was allowed to stand for 1 hour at room temperature and concentrated to dryness under reduced pressure. 4 mL of toluene was added to the residue and again concentrated to dryness under reduced pressure. In this way (S)-3-aminooctanoylglycine benzyl ester was produced. To this compound was added 155.3 mg of Boc-(2R,3S)-3-amino-2-methyloctanoic acid, 266 mg of Bop reagent and 82 mg of HOBt, and the mixture was dissolved in 2 mL of DMF. 257 μL of triethylamine was added in ice-cold conditions with stirring, the temperature of the resultant solution was brought back to room temperature 30 minutes after the addition, and the solution was stirred for 16 hours. 50 mL of ethyl acetate was added to the reaction solution, which was then placed in a separating funnel. The reaction solution was then washed with equal volumes of 10% citric acid aqueous solution, 4% sodium acid carbonate aqueous solution, water, and saturated salt water, dried for 1 hour with anhydrous sodium sulfate, and concentrated to dryness under reduced pressure to yield 292 mg of Boc-(2R,3S)-3-amino-2-methyloctanoyl-(S)-4-aminooctanoyl-glycine benzyl ester. This compound was then dissolved in 4 mL of trifluoroacetic acid, and the solution was allowed to stand for 1 hour at room temperature and concentrated to dryness under reduced pressure. The resultant compound was dissolved in 10 mL of methanol, and 20 mg of palladium black was added to the solution and stirred for 1 hour in a hydrogen stream for hydrogenolysis of benzyl ester. The catalyst was filtered off, and the solution was concentrated to dryness under reduced pressure, and the residue was dissolved in a 100:20:1 mixture of chloroform, methanol and ammonia water, and the mixture was poured into a 100 mL-silica gel column. Subsequently, the column was eluted with a 100:20:1 mixture solvent of chloroform, methanol and ammonia water, then with a 100:30:1 mixture solvent of chloroform, methanol and ammonia water for purification of a compound of interest. The analytical values are shown below.

¹H NMR (DMSO-6d, 600 MHz)

δ 0.85 (3H, t, J=7.2), 0.87 (3H, t, J=7.2), 1.07 (3H, d, J=7.0), 1.26 (1H, m), 1.37 (2H, m), 1.47 (3H, m), 2.27 (2H, d, J=6.9), 2.50 (1H, m), 3.20 (1H, m), 3.73 (2H, AB type) q-like, 4.04 (1H, m), 7.80 (3H, br d), 8.00 (1H, d, J=8.5), 8.18 (1H, t, J=5.9),

### Example 2

### Production of (S)-3-aminohexyl-(S)-3-aminooctanoyl-glycine

Boc-(3S)-3-aminohexanoic acid was prepared as follows and Boc-(S)-3-aminooctanoyl-glycine benzyl ester was prepared as in Example 1. Using these compounds, (S)-3-aminohexyl-(S)-3-aminooctanoyl-glycine was produced in the manner described below.

### [1] Preparation of Boc-(3S)-3-aminohexanoic acid

(S)-3-aminohexanoic acid was prepared as in [2-1] of Example 1 for preparation of (S)-3-aminooctanoic acid except that ethyl 2-hexenoate was employed instead of ethyl 2-octenoate. Subsequently, using Boc₂O, Boc-(3S)-3-aminohexanoic acid was prepared as in [2-2] of Example 1.

### [2] Production of (S)-3-aminohexyl-(S)-3-aminooctanoyl-glycine

(S)-3-aminohexyl-(S)-3-aminooctanoyl-glycine was produced in a manner similar to that described in Example 1 by processing Boc-(S)-3-aminooctanoylglycine benzyl ester obtained as in Example 1 and Boc-(35)-3-aminohexanoic acid obtained in [1]. The analytical values are shown below.

¹H NMR (D₂O, 600 MHz)

δ 0.85 (3H, t, J=7.2), 0.87 (3H, t, J=7.2), 1.20, 1.25, 1.34 (9H, m), 1.48 (3H, m), 2.28 (2H, AB type), 2.35 (1H, dd, J=6.8, 15.4), 2.44 (1H, dd, J=5.9, 15.5), 3.37 (1H, m), 3.73 (2H, AB type), 4.08 (1H, m), 7.79 (3H, br.s), 8.00 (1H, d, J=8.6), 8.22 (1H, t, J=5.9)

### Example 3

### Production of (S)-3-amino-5-methylhexyl-(S)-3-aminooctanoyl-glycine

(S)-3-amino-5-methylhexyl-(S)-3-aminooctanoyl-glycine was produced in a manner similar to that described in Example 1 by processing Boc-(S)-3-aminooctanoyl-glycine benzyl ester prepared as in [2-3] of Example 1 and Boc-(3S)-3-amino-5-methylhexanoic acid

(from Aldrich, Boc-β-homoleucine). The analytical values are shown below.

¹H NMR (D₂O, 600 MHz)

δ 0.86 (9H, m), 1.20-1.25 (7H, m), 1.32 (1H, m), 1.42 (1H, m), 1.46 (1H, m), 1.79 (1H, m), 2.28 (2H, AB type), 2.35 (1H, dd, J=6.4, 15.4), 2.43 (1H, dd, J=5.9, 15.3), 3.41 (1H, m), 3.73 (2H, AB type), 4.08 (1H, m), 7.77 (3H, br.s), 8.01 (1H, d, J=8.6), 8.22 (1H, t, J=5.9)

### Example 4

### Production of (S)-3-aminooctanoyl-(S)-3-aminooctanoyl-glycine

Using Boc-(S)-3-aminooctanoyl-(S)-3-aminooctanoic acid prepared below and glycine t-butyl ester hydrochloride, (S)-3-aminooctanoyl-(S)-3-aminooctanoyl-glycine was produced in the manner described below.

### [1] Preparation of Boc-(S)-3-aminooctanoyl-(S)-3-aminooctanoic acid

### [1-1] Preparation of ethyl (S)-3-aminooctanoate hydrochloride

In the preparation of (S)-3-aminooctanoic acid in [2-1] of Example 1, the product obtained in the reduction step was not hydrolyzed, one equivalent amount of hydrogen chloride/dioxane solution was added thereto, and the resultant solution was concentrated, dissolved in chloroform, and purified on a silica gel column using a 15:1 mixture of chloroform and methanol. In this way ethyl (S)-3-aminooctanoate hydrochloride was obtained as a slightly colored, hygroscopic solid.

### [1-2] Preparation of ethyl Boc-(S)-3-aminooctanoyl-(S)-3-aminooctanoate

260 g of ethyl (S)-3-aminooctanoate hydrochloride obtained in [1-1], 251 mg of Boc-(S)-3-aminooctanoic acid prepared as in [1-2] of Example 1, 471 mg of Bop reagent, and 144 mg of HOBt were dissolved in 3 mL of DMF, 434 μL of triethylamine was added, and stirred for 16 hours. The reaction solution was then washed with acid and alkali through a normal method and dried, and then concentrated to dryness to yield crude ethyl Boc-(S)-3-aminooctanoyl-(S)-3-aminooctanoate. This compound was then purified on a silica gel column using chloroform. The analytical values are shown below.

¹H NMR (4d-MeOH, 600 MHz)δ; 0.90 (3H, t, J=7.0 Hz), 1.24 (3H, t, J=7.2 Hz), 1.32 (13H, m,), 1.43 (9H, s, Boc), 1.4-1.55 (3H, m), 2.26 (1H, dd, J=13.8, 7.0), 2.31 (1H, dd, J=6.8, 13.9), 2.45 (2H, AB type m), 3.81 (1H, m), 4.11 (2H, dq, J=7.1, 2.0 Hz), 4.18 (1H, m),

### [1-3] Preparation of Boc-(S)-3-aminooctanoyl-(S)-3-aminooctanoic acid

248 mg of ethyl Boc-(S)-3-aminooctanoyl-(S)-3-aminooctanoate obtained in [1-2] was dissolved in 8 mL of methanol, 582 μL of 5 N NaOH was added, and stirred. Seven hours afterward, the solution was neutralized by the addition of 582 μL of 5 N hydrochloric acid and concentrated, and dissolved in a small amount of methanol. The solution was purified on a 200 mL-column packed with Sephadex LH20 to yield Boc-(S)-3-aminooctanoyl-(S)-3-aminooctanoic acid. The analytical values are shown below.

¹H NMR (4d-MeOH, 600 MHz)

δ 0.90 (3H, t, J=7.1 Hz), 1.32 (13H, m,), 1.43 (9H, s, Boc), 1.47 (2H, m), 1.55 (1H, m), 2.27 (1H, dd, J=13.9, 7.0), 2.32 (1H, dd, J=6.9, 13.9), 2.42 (1H, dd, J=15.6, 6.8), 2.46 (1H, dd, J=6.5, 15.7), 3.82 (1H, m), 4.17 (1H, m)

¹H NMR (CDCl₃, 600 MHz)

δ 0.86 (6H, t-like, J=6.7 Hz), 1.28 (14H, m), 1.42 (9H, s, Boc), 1.54 (2H, m), 2.40 (2H, m), 2.54 (2H, m), 3.81 (1H, m), 4.21 (1H, m), 4.06 (1H, br.s), 6.49 (1H, d, J=14.6 NH)

### [2] Production of (S)-3-aminooctanoyl-(S)-3-aminooctanoyl-glycine

238 mg of Boc-(S)-3-aminooctanoyl-(S)-3-aminooctanoic acid obtained in [1], 1120 mg of glycine t-butyl ester hydrochloride (Aldrich, Cat. No. 347957-5G), 290 mg of Bop reagent, and 89 mg of HOBt were dissolved in 2 mL of DMF, 267 μL of triethylamine was added, and stirred for 16 hours. The reaction solution was then washed with acid and alkali through a normal method and dried, and then concentrated to dryness to yield crude Boc-(S)-3-aminooctanoyl-(S)-3-aminooctanoyl-glycine t-butyl ester. This compound was dissolved in 4 mL of trifluoroacetic acid, stirred for 1 hour at room temperature, and concentrated to dryness. The resultant compound was then purified on a silica gel column using a 100:20:1 mixture of chloroform, methanol and ammonia water, and then using a 100:30:1 mixture thereof. The analytical values are shown below.

¹H NMR (DMSO-6d+TFA, 600 MHz)

δ 0.85 (3H, t, J=7.1 Hz), 0.87 (3H, t, J=7.1 Hz), 1.26 (13H, m,), 1.48 (3H, m), 2.28 (2H, m AB type), 2.35 (1H, dd, J=6.6, 15.4), 2.44 (1H, dd, J=6.1, 15.5), 3.37 (1H, m), 3.73 (2H, m AB type), 4.08 (1H, m), 7.78 (3H, br.s, NH3⁺), 8.00 (1H, d, J=8.6, NH), 8.21 (1H t, J=5.8, NH)

¹³C NMR (DMSO-6d+TFA, 150 MHz)

δ 13.8, 13.9, 21.9, 22.1, 24.1, 25.1, 30.0, 31.2, 32.0, 33.5, 37.2, 40.6, 48.4, 168.7, 170.4, 171.4

### Production of (S)-3-aminooctanoyl-(S)-3-aminooctanoyl-sarcosine

(S)-3-aminooctanoyl-(S)-3-aminooctanoyl-sarcosine was produced in a manner similar to that described in Example 4 except that sarcosine t-butyl ester hydrochloride (Aldrich, 460613-5G) was employed instead of glycine t-butyl ester hydrochloride. The analytical values are shown in below.

¹H NMR (DMSO-6d+TFA, 600 MHz)

δ 0.85 (3H, t, J=7.1 Hz), 0.87 (3H, t, J=7.1 Hz), 1.26 (13H, m,), 1.50 (3H, m), 2.28+2.43 (2H, m AB type), 2.35 (1H m), 2.44 (1H, dd, J=6.1, 15.5), 2.52 (1H, m), 2.81+3.01 (3H, s, N—CH₃, cis and trans), 3.37 (1H, m), 3.97 (1H, dd, J=17.1, 40.0), 4.13 (1H, dd, J=18.6, 35.0), 7.79 (3H br.s, NH3⁺), 8.01 (1H, dd, J=8.5, 21.6 NH)

### Example 6

### Production of (S)-3-aminooctanoyl-(S)-3-aminooctanoyl-L-alanine

(s)-3-aminooctanoyl-(s)-3-aminooctanoyl-sarcosine was produced in a manner similar to that described in Example 4 except that L-alanine t-butyl ester hydrochloride (Kokusan Chemical Co., Ltd., commodity code: 251704) was employed instead of glycine t-butyl ester hydrochloride. The analytical values are shown in below.

¹H NMR (DMSO-6d+TFA, 600 MHz)

δ 0.85 (3H, t, J=7.1 Hz), 0.87 (3H, t, J=7.1 Hz), 1.26 (13H, m,), 1.26 (3H, d, J=7.3 Hz, Ala-CH₃), 1.48 (3H, m), 2.26 (2H, m AB type), 2.35 (1H, dd, J=6.2, 15.4 Hz), 2.43 (1H, dd, J=6.2, 15.4 Hz), 3.37 (1H, m), 4.08 (1H, m), 4.19 (1H, q, J=7.3 Hz), 7.79 (3H, br.s, NH3+), 8.00 (1H, d, J=8.8 Hz, NH), 8.18 (1H, d, J=7.2 Hz)

### Example 7

### Production of (S)-3-aminooctanoyl-(S)-3-aminooctanoyl-β-alanine

(S)-3-aminooctanoyl-(S)-3-aminooctanoyl-β-alanine was produced in a manner similar to that described in Example 4 except that β-alanine t-butyl ester hydrochloride was employed instead of glycine t-butyl ester hydrochloride.

The β-alanine t-butyl ester was prepared as an oily substance as follows: 1.76 g of t-butyl ester was dissolved in 16 mL of dioxane, 4 mL of condensed ammonia water was added and stirred for 20 hours at room temperature; thereafter, the solution was concentrated under reduced pressure and purified by silica gel chromatography (20:1 mixture of chloroform and methanol). The analytical values of (S)-3-aminooctanoyl-(S)-3-aminooctanoyl-β-alanine are shown below.

¹H NMR (DMSO-6d+TFA, 600 MHz)

δ 0.85 (3H, t, J=7.1 Hz), 0.87 (3H, t, J=7.1 Hz), 1.25 (13H, m,), 1.47 (3H, m), 1.91 (2H, s, CH₂CH₂COOH), 2.28 (2H, m AB type), 2.35 (1H, dd, J=6.4, 15.2 Hz), 2.35 (1H, dd, J=6.3, 15.4 Hz), 3.37 (1H, m), 3.73 (2H, m, AB type CH₂CH₂COOH), 4.07 (1H, m), 7.78 (3H, br.s, NH3⁺), 7.99 (1H, d, J=8.6 Hz NH), 8.21 (1H, t, J=5.9 Hz)

### Example 8

### Production of (S)-3-aminooctanoyl-(S)-3-aminooctanoyl-L-proline

(S)-3-aminooctanoyl-(S)-3-aminooctanoyl-L-proline was produced in a manner similar to that described in Example 4 except that L-proline t-butyl ester (Kokusan Chemical Co., Ltd., commodity code: 2517302) was employed instead of glycine t-butyl ester hydrochloride. The analytical values are shown below.

¹H NMR (DMSO-6d+TFA, 600 MHz)

δ 0.85 (3H, t, J=7.0), 0.87 (3H, t, J=7.0), 1.25 (13H, m), 1.51 (3H, m), 1.7-2.5 (m, 8H), 3.37-3.52 (3H), 4.09 (1H, m), 4.20 (0.75H, dd, J=3.9, 8.8), 4.49 (0.25H, dd, J=2.5, 8.8), 7.78 (3H, br.s), 8.02 (0.25H, d, J=8.6), 8.05 (0.75H, d, J=8.7),

### Example 9

### Production of (S)-3-aminooctanoyl-(S)-3-aminooctanoyl-L-valine

(S)-3-aminooctanoyl-(S)-3-aminooctanoyl-L-valine was produced in a manner similar to that described in Example 4 except that L-valine t-butyl ester hydrochloride (Kokusan Chemical Co., Ltd., commodity code: 2517370) was employed instead of glycine t-butyl ester hydrochloride. The analytical values are shown below.

¹H NMR (DMSO-6d+TFA, 600 MHz)

δ 0.85 (3H, t, J=7.1), 0.88 (9H, m), 1.25 (13H, m), 1.42 (1H, m), 1.49 (2H, m), 2.03 (1H, m) Val-3, 2.27 (1H, dd, J=6.1, 14.0), 2.36 (2H, m), 2.44 (1H, dd, J=6.2, 15.4), 3.37 (1H, m), 4.09 (1H, m), 4.16 (1H, dd, J=5.9, 8.5), 7.79 (3H, br.s), 8.00 (1H, d, J=8.6), 8.04 (1H, d, J=8.6),

### Example 10

### Production of (S)-3-aminooctanoyl-(S)-3-aminooctanoyl-L-leucine

(S)-3-aminooctanoyl-(S)-3-aminooctanoyl-L-leucine was produced in a manner similar to that described in Example 4 except that L-leucine t-butyl ester hydrochloride (Kokusan Chemical Co., Ltd., commodity code: 2517728) was employed instead of glycine t-butyl ester hydrochloride. The analytical values are shown below.

¹H NMR (DMSO-6d+TFA, 600 MHz)

δ 0.84 (3H, d, J=6.7), 0.85 (3H, t, J=7.3), 0.87 (3H, t, J=7.1), 0.90 (3H, d, J=6.6), 1.25 (13H, m), 1.41 (1H, m), 1.51 (4H, m), 1.64 (1H, m), 2.27 (2H, m), 2.34 (1H, dd, J=6.5, 15.3), 2.44 (1H, dd, J=6.2, 15.3), 3.37 (1H, m), 4.08 (1H, m), 4.23 (1H, m), 7.79 (3H, br d, J=4.0), 8.01 (1H, d, J=8.6), 8.16 (1H, d, J=8.1),

### Example 11

### Production of (S)-3-aminooctanoyl-(S)-3-aminooctanoyl-L-phenylalanine

(S)-3-aminooctanoyl-(S)-3-aminooctanoyl-L-phenylalanine was produced in a manner similar to that described in Example 4 except that L-phenylalanine t-butyl ester hydrochloride (Kokusan Chemical Co., Ltd., commodity code: 2517256) was employed instead of glycine t-butyl ester hydrochloride. The analytical values are shown below.

¹H NMR (DMSO-6d+TFA, 600 MHz)

δ 0.84 (3H, t, J=7.2), 0.87 (3H, t, J=7.1), 1.09+1.17+1.22+1.27 (14H, m), 1.48 (2H, m), 2.19 (1H, dd, J=8.5, 14.1), 2.23 (1H, dd, J=5.5, 14.1), 2.33 (1H, dd, J=6.6, 15.4), 2.41 (1H, dd, J=6.2, 15.4), 2.85 (1H, dd, J=9.7, 13.9) Phe-b, 3.07 (1H, dd, J=4.8, 13.9) Phe-b, 3.36 (1H, m), 3.99 (1H, m), 4.48 (1H, m), 7.24 (5H, m), 7.78 (3H, br d, J=3.8), 7.94 (1H, d, J=8.7), 8.23 (1H, t, J=8.3),

### Example 12

### Production of (S)-3-aminooctanoyl-(S)-3-aminodecanoyl-glycine

Boc-(S)-3-aminooctanoyl-(S)-3-aminodecanoic acid was first prepared from Boc-(S)-3-aminooctanoic acid obtained in [1-2] of Example 1 and ethyl (S)-3-aminodecanoate prepared as described below. Subsequently, (S)-3-aminooctanoyl-(S)-3-aminodecanoyl-glycine was produced in a manner similar to that described in Example 4 using Boc-(S)-3-aminooctanoyl-(S)-3-aminodecanoic acid and glycine t-butyl ester hydrochloride.

### Preparation of ethyl (S)-3-aminodecanoate

In the preparation of (S)-3-aminodecanoic acid of Example 1, ethyl 2-decenoate (Tokyo Chemical Industry Co., Ltd., commodity code: D2767) was employed instead of 2-octenoic acid for addition of chiral amine, followed by contact reduction to yield ethyl 3-aminodecanoate. By treatment with a calculated amount of hydrochloric acid, ethyl 3-aminodecanoate in hydrochloride form was obtained, which was then purified by silica gel chromatography (15:1 mixture of chloroform and methanol).

The analytical values of (S)-3-aminooctanoyl-(S)-3-aminodecanoyl-glycine are as follows:

¹H NMR (D₂O, 600 MHz)

δ 0.86 (3H, t, J=7.1), 0.87 (3H, t, J=7.1), 1.24 (17H, m), 1.48 (3H, m), 2.28 (2H, AB type), 2.35 (1H, dd, J=6.5, 15.4), 2.43 (1H, dd, J=6.3, 15.4), 3.38 (1H, m), 3.73 (2H, AB type), 4.07 (1H, m), 7.79 (3H, br.s), 7.99 (1H, d, J=8.5), 8.21 (1H, t, J=5.9)

### Example 13

### Production of (S)-3-aminooctanoyl-(S)-3-aminodecanoyl-L-proline

(S)-3-aminooctanoyl-(S)-3-aminodecanoyl-L-proline was produced in a manner similar to that described in the preparation of (S)-3-aminooctanoyl-(S)-3-aminodecanoyl-glycine of Example 12 except that L-proline t-butyl ester was employed instead of glycine t-butyl ester hydrochloride. The analytical values are shown below.

¹H NMR (D₂O, 600 MHz)

δ 0.86 (3H, t, J=7.2), 0.87 (3H, t, J=7.1), 1.24 (17H, m), 1.51 (3H, m), 1.7-2.36 (m, 7H), 2.44 (1H, dd, J=5.8, 15.3), 3.37-3.52 (3H), 4.08 (1H, m), 4.20 (0.75H, dd, J=3.9, 8.7), 4.49 (0.25H, dd, J=2.5, 8.5), 7.79 (3H, br.s), 8.01 (0.25H, d, J=8.6), 8.04 (0.75H, d, J=8.6),

### Example 14

### Evaluation of Inhibition Activity of Aflatoxin Production

### —Preparation of Spore Suspension of Aflatoxin-Producing Microorganism—

As the aflatoxin-producing microorganism, Aspergillus parasiticus NRRL 2999 was cultured on slant potato dextrose agar medium (PDA medium, NISSUI PHARMACEUTICAL CO., LTD.) for 14 days at 27° C., and spores were scraped off from the flora with a platinum loop and suspended in 0.01% Tween 80 (Sigma) aqueous solution to prepare a spore suspension.

The spore suspension was diluted and spread over the PDA medium, and cultured for 2 days. The number of emerged colonies was taken as the number of spores.

### —Assay of Inhibition Activity of Aflatoxin Production—

The dioctatin derivatives (aflatoxin production inhibitors) prepared in Examples 1, 4, 5, 6, 7, 8 and 12 were each added in 1 mL of autoclaved PD liquid medium (DIFCO) in a sterile manner to prepare dioctatin derivative samples with increasing concentrations

(from 0 to 20 μg/mL). The samples were inoculated with 10 μL of the spore suspension (1.9×10⁴ CFU) and incubated for 3 days at 27° C. Here, a 24-well plate (IWAKI) was used for incubation, and each aflatoxin production inhibitor was dissolved in methanol-hydrochloric acid (volume ratio=100:0.009) solution and added in a volume of 20 μL.

The culture from each well (50 μL) was diluted 1,000-fold with distilled water, and the amount of aflatoxin (total amount of aflatoxins B₁, B₂, G₁, and G₂) contained in 50 μL of each diluted culture solution was quantified using a commercially available ELISA kit (RIDASCREEN FAST Aflatoxin, from R-Biopharm AG). The assay was done in triplicate. Inhibition ratio (%) was calculated for each concentration of inhibitor using the following equation:

Inhibition ratio (%)=[(A)−(B)]/(A)×100]

where (B) denotes the average of aflatoxin amounts of three culture samples, and (A) denotes the aflatoxin amount of the inhibitor-free sample.

The values of IC₅₀ (50% inhibition concentration) were calculated based on the inhibition ratio (%) for each concentration. The results are shown in Table 2.

As a reference, the assay results for dioctatin A (Reference Example 1) and dioctatin B (Reference Example 2), which are naturally occurring dioctatins chemically synthesized and which have three-dimensional structure, are also shown in Table 2.

TABLE 2

| | | IC₅₀ |
|---|---|---|
| | Compound | (μg/mL) |
| Example 1 | (2R,3S)-3-amino-2-methyloctanoyl-(S)-4-aminooctanoyl-glycine | 6.6 |
| Example 4 | (S)-3-aminooctanoyl-(S)-3-aminooctanoyl-glycine | 2.5 |
| Example 5 | (S)-3-aminooctanoyl-(S)-3-aminooctanoyl-sarcosine | 2.9 |
| Example 6 | (S)-3-aminooctanoyl-(S)-3-aminooctanoyl-L-alanine | 1.4 |
| Example 7 | (S)-3-aminooctanoyl-(S)-3-aminooctanoyl-β-alanine | 3.7 |
| Example 8 | (S)-3-aminooctanoyl-(S)-3-aminooctanoyl-L-proline | 0.5 |
| Example 12 | (S)-3-aminooctanoyl-(S)-3-aminodecanoyl-glycine | 4.0 |
| — | dioctatin A (Reference Example 1) | 4.6 |
| — | dioctatin B (Reference Example 2) | 5.1 |

The results of Table 2 demonstrate that the dioctatin derivatives of the present invention have excellent aflatoxin production inhibition activity like naturally occurring dioctatins, and that the glycine-substituted dioctatin derivative prepared in Example 4 and the L-amino acid residue-substituted dioctatin derivatives prepared in Examples 6 and 8 all have extremely excellent aflatoxin production inhibition activity.

### INDUSTRIAL APPLICABILITY

The dioctatin derivatives of the present invention are useful aflatoxin production inhibitors, and production of aflatoxin can be readily inhibited by application to various targets to which aflatoxin-producing microorganism is attached or which is infected with the aflatoxin-producing microorganism. Moreover, the dioctatin derivatives of the present invention can be suitably used for a method of controlling aflatoxin contamination of the present invention, particularly for a method of controlling aflatoxin contamination directed to vegetables and farm crops.

What is claimed is:

1. Dioctatin derivatives represented by the following Structural Formula (I): 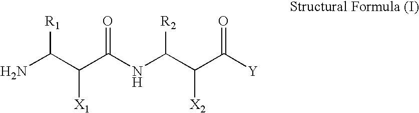
where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; and Y represents 2-amino-2-butenoic acid or amino acid residue, with compounds where R₁ and R₂ are each CH₃(CH₂)₄—, X₂ is a hydrogen atom and Y is 2-amino-2-butenoic acid being excluded.

2. The dioctatin derivatives according to claim 1, wherein the dioctatin derivatives are represented by the following Structural Formula (II): 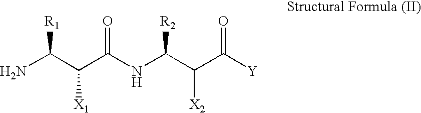
where R₁ and R₂ each represent CH₃—(CH₂)ₙ—, (CH₃)₂CH—CH₂— or C₆H₅—CH₂—; n represents an integer of 2 to 6; X₁ represents a hydrogen atom or CH₃; X₂ represents a hydrogen atom; and Y represents 2-amino-2-butenoic acid or amino acid residue, with compounds where R₁ and R₂ are each CH₃(CH₂)₄— and Y is 2-amino-2-butenoic acid being excluded.

3. The dioctatin derivatives according to claim 1, wherein the amino acid residue is selected from the group consisting of glycine residue, sarcosine residue, L-alanine residue, β-alanine residue, L-proline residue, L-valine residue, L-leucine residue, L-phenylalanine residue, L-thioproline residue, and 4-hydroxy-L-proline residue.

4. A production process of dioctatin derivative comprising:
condensing a compound represented by the following Structural Formula (b) with an amino acid derivative in which its carboxyl group is protected, to prepare a dipeptide compound;
removing a protective group of an amino group of the dipeptide compound;
condensing the dipeptide compound with a compound represented by the following Structural Formula (a) to prepare a tripeptide compound; and
removing a protective group of the tripeptide compound. 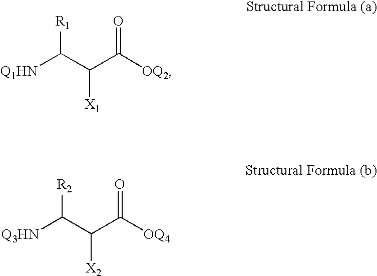
where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ and Q₃ each represent Boc group, carbobenzoxy group, p-methoxybenzyloxycarbonyl group, Fmoc group, 2,2,2-trichloroethoxycarbonyl group, or allyloxycarbonyl group; and Q₂ and Q₄ each represent a hydrogen atom.

5. A production process of dioctatin derivative comprising:
condensing a compound represented by the following Structural Formula (a) with a compound represented by the following Structural Formula (b), to prepare a dipeptide compound;
removing a protective group of a carboxyl group of the dipeptide compound;
condensing the dipeptide compound with an amino acid derivative in which its carboxyl group is protected, to prepare a tripeptide compound; and
removing a protective group of the tripeptide compound. 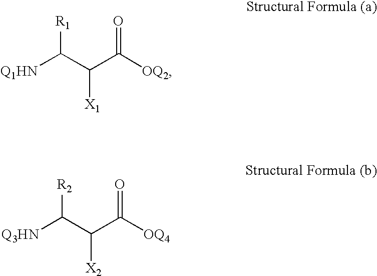
where R₁ and R₂ each represent CH₃—(CH₂)ₙ—, (CH₃)₂CH—CH₂— or C₆H₅—CH₂—; n represents an integer of 2 to 6; X₁ and X₂ each represent a hydrogen atom or CH₃; Q₁ represents Boc group, carbobenzoxy group, p-methoxybenzyloxycarbonyl group, Fmoc group, 2,2,2-trichloroethoxycarbonyl group, or allyloxycarbonyl group; Q₂ and Q₃ each represent a hydrogen atom; and Q₄ represents a hydrogen atom, methyl group, ethyl group, benzyl group, t-butyl group, or 2,2,2-trichloroethyl group.

6. The production process according to claim 4, wherein the compounds represented by Structural Formulas (a) and (b) have S configuration at 3-position.

7. The production process according to claim 5, wherein the compounds represented by Structural Formulas (a) and (b) have S configuration at 3-position.

8. The production process according to claim 4, wherein in Structural Formula (a) X₁ represents CH₃, and the compounds represented by Structural Formula (a) have R configuration.

9. The production process according to claim 5, wherein in Structural Formula (a) X₁ represents CH₃, and the compounds represented by Structural Formula (a) have R configuration.

10. The production process according to claim 4, wherein the amino acid derivative is selected from the group consisting of glycine, sarcosine, L-alanine, β-alanine, L-proline, L-valine, L-leucine, L-phenylalanine, L-thioproline, and 4-hydroxy-L-proline.

11. The production process according to claim 5, wherein the amino acid derivative is selected from the group consisting of glycine, sarcosine, L-alanine, β-alanine, L-proline, L-valine, L-leucine, L-phenylalanine, L-thioproline, and 4-hydroxy-L-proline.

12. An aflatoxin production inhibitor comprising a dioctatin derivative represented by the following Structural Formula (I): 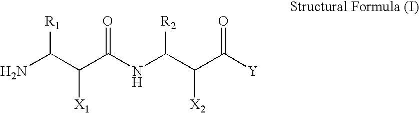
where R₁ and R₂ each represent CH₃—(CH₂)ₙ—, (CH₃)₂CH—CH₂— or C₆H₅—CH₂—; n represents an integer of 2 to 6; X₁ and X₂ each represent CH₃ or hydrogen atom; and Y represents 2-amino-2-butenoic acid or amino acid residue, with a compound where R₁ and R₂ are each CH₃(CH₂)₄—, X₂ is a hydrogen atom and Y is 2-amino-2-butenoic acid being excluded.

13. The aflatoxin production inhibitor according to claim 12, wherein the dioctatin derivative is represented by the following Structural Formula (III): 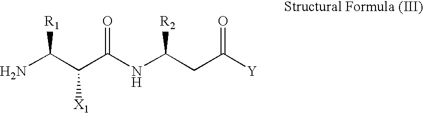
where R₁ and R₂ each represent CH₃—(CH₂)ₙ— or (CH₃)₂CH—CH₂—; X₁ represents a hydrogen atom or CH₃; n represents an integer of 2 to 6; and Y represents an amino acid residue.

14. A method of aflatoxin contamination control comprising:
inhibiting aflatoxin production by aflatoxin-producing microorganism by use of an aflatoxin production inhibitor comprising a dioctatin derivative represented by the following Structural Formula (I): 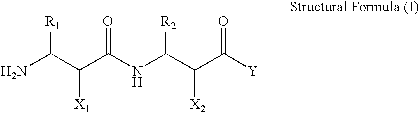
where R₁ and R₂ each represent CH₃—(CH₂)ₙ—, (CH₃)₂CH—CH₂— or C₆H₅—CH₂—; n represents an integer of 2 to 6; X₁ and X₂ each represent CH₃ or hydrogen atom; and Y represents 2-amino-2-butenoic acid or amino acid residue, with a compound where R₁ and R₂ are each CH₃(CH₂)₄—, X₂ is a hydrogen atom and Y is 2-amino-2-butenoic acid being excluded.

15. The method of aflatoxin contamination control according to claim 14, wherein the aflatoxin production inhibitor is applied to a farm crop for inhibition of production of aflatoxin from aflatoxin-producing microorganism that infected the crop.
